(19) United States
(12) Patent Application Publication
(10) Pub. No.: US 2010/0260824 A1
(43) Pub. Date: Oct. 14, 2010

(54) Antimicrobial Foam Compositions, Articles and Methods

(75) Inventors: Chirag B. Shah, North Attleboro, MA (US); Ronald F. Vitaris, Worcester, MA (US); E. David Fink, Franklin, MA (US); Scott Orr, Franklin, MA (US); Brian Dowd, Dedham, MA (US); Alain Tranchemontagne, Warwick, RI (US); Kate Ward, Marshfield, MA (US); Hansen Swaniker, Tallahassee, FL (US); Harish A. Patel, Norfolk, MA (US)

Correspondence Address: TYCO HEALTHCARE GROUP LP, 15 HAMPSHIRE STREET, MANSFIELD, MA, 02048, US

(73) Assignee: Tyco Healthcare Group LP, Mansfield, MA (US)

(21) Appl. No.: 12/548,532

(22) Filed: Aug. 27, 2009

Related U.S. Application Data
(60) Provisional application No. 61/136,334, filed on Aug. 28, 2008.
(60) Provisional application No. 61/136,335, filed on Aug. 28, 2008.

(51) Int. Cl. A61L 15/16; A61K 31/785; A61K 38/02; A61P 17/02

(52) U.S. Cl. 424/447; 424/78.08; 514/1.1

(57) ABSTRACT

Articles may be formed including: at least one layer of foam, the foam layer and at least one antimicrobial agent associated with foam layer, the antimicrobial agent including PHMB, PEHMB, or derivatives thereof; at least one non-adherent layer disposed on at least a portion of the foam layer, the non-adherent layer being permeable to moisture; and a film disposed on at least another portion of the foam layer, the film being breathable to allow escape of moisture, but substantially impermeable to bacteria. Another article may include at least one layer of foam, the foam having pores of different sizes, at least some of the pores at least partially filled with at least one elutable antimicrobial agent, the pores of different sizes forming a gradient with the foam layer. Yet another article may include a foam matrix and a plurality of dissolvable members disposed with the foam matrix, at least one antimicrobial agents associated with the dissolvable members such that upon dissolution thereof the antimicrobial agent is eluted and pores or voids are created in the foam matrix. Wound dressings formed from the above articles are also described.

### FIELD

The present invention is directed to antimicrobial foam compositions, articles and methods.

### BACKGROUND

In this specification where a document, act or item of knowledge is referred to or discussed, this reference or discussion is not an admission that the document, act or item of knowledge or any combination thereof was at the priority date, publicly available, known to the public, part of common general knowledge, or otherwise constitutes prior art under the applicable statutory provisions; or is known to be relevant to an attempt to solve any problem with which this specification is concerned.

A variety of anti-microbial compositions, articles and methods have been suggested. However, such wound compositions and methods possess various deficiencies and shortcomings.

For example, U.S. Pat. No. 5,465,735 appears to disclose wound dressings comprising an absorbent pad for receiving and retaining wound fluids sandwiched between first and second outer sheet materials, the first sheet material for placement on the wound being a perforated non-adherent film for preventing the dressing from sticking to the wound, the second sheet material being characterized as being bacteria-impermeable, the absorbent pad being a multilayer structure comprising an inner layer of a low density absorbent material for receiving fluids diffusing to the dressing from the wound and an overlying layer of a high density absorbent material for receiving and retaining wound fluids diffusing through the inner layer in order to inhibit skin maceration due to the wetness of the surface area of the absorbent pad adjacent the wound.

However, a need still exists in the art for compositions, devices and methods which have increased effectiveness in reducing and/or preventing development of unwanted microbial organisms, are safe, and provide for improved efficiencies in wound care management.

While certain aspects of conventional technologies have been discussed to facilitate disclosure of the invention, Applicants in no way disclaim these technical aspects, and it is contemplated that the claimed invention may encompass one or more of the conventional technical aspects discussed herein.

### DEFINITIONS

As used herein, unless otherwise indicated, the terms “microbial organism” or “microbial” will be used to refer to microscopic organisms of matter, including fungal, bacterial and/or viral organisms. Thus, the term “antimicrobial” as used herein refers to a composition or agent that kills or otherwise inhibits the growth of such fungal, bacterial and/or viral organisms.

### SUMMARY

The present invention may address one or more of the problems and deficiencies of the prior art discussed above. However, it is contemplated that the invention may prove useful in addressing other problems and deficiencies, or provide benefits and advantages, in a number of technical areas. Therefore the claimed invention should not necessarily be construed as limited to addressing any of the particular problems or deficiencies discussed herein.

The present invention may optionally possess one or more of the following features, benefits or advantages: an absorbent foam with antimicrobial properties, wherein wound exudate is held in a chamber away from the wound and skin thereby preventing skin maceration and the spread of bacteria in the wound; spiral cut foam allowing for improved wound contact and fluid management; a foam material with a porosity gradient to facilitate controlled release of an agent into a wound; a foam material having a porosity gradient including at least one agent to deliver a relatively large amount of agent initially, followed with the delivery of a decreasing amount of agent with time; a foam material having a porosity gradient including at least one agent to deliver a relatively small amount of agent initially, followed by the delivery of a larger amount of agent with time; a foam material having a porosity gradient such that smaller pores are proximate to the wound-facing side of the material, and relatively larger pores located further into the material away from the wound-facing side; a foam material having a porosity gradient such that larger pores are located proximate to the wound-facing side of the material, and relatively smaller pores are located further into the material away from the wound-facing side; a foam material having a porosity tailored such that the presence of relatively high levels of exudate will prompt delivery of relatively large amounts of agent to the wound; and a foam material having a porosity tailored such that the presence of relatively low levels of exudate will prompt delivery of relatively small amounts of agent to the wound; dissolvable beads of varying size containing an antimicrobial agent and embedded in foam not only act as a carrier and delivery matrix for the antimicrobial agent, but also because of varying bead size an/or concentration gradient(s), deliver different concentrations of the antimicrobial agent at different times as needed; dissolvable beads embedded in foam present a dressing that has on-demand absorptive capacity; controlling pH to a slightly acidic level in the range of 6 to 7 to reduce chance of wound infections.

According to one alternative aspect, the present invention provides an article comprising: at least one layer of foam, and at least one antimicrobial agent associated with foam layer, the antimicrobial agent comprising PHMB, PEHMB or derivatives thereof; at least one non-adherent layer disposed on at least a portion of the foam layer, the non-adherent layer being permeable to moisture; and a film disposed on at least another portion of the foam layer, the film being breathable to allow escape of moisture, but substantially impermeable to bacteria.

According to a further aspect, the present invention provides an article comprising at least one layer of foam, the foam comprising pores of different sizes, at least some of the pores at least partially filled with at least one elutable antimicrobial agent, the pores of different sizes forming a gradient with the foam layer.

According to yet another aspect, the present invention provides an article comprising: a foam matrix and a plurality of dissolvable members disposed with the foam matrix, at least one antimicrobial agent associated with the dissolvable members such that upon dissolution thereof the antimicrobial agent is eluted and pores or voids are created in the foam matrix.

### BRIEF DESCRIPTION OF THE DRAWINGS

FIG. 1 is a schematic sectional view of an article, composition, laminate or dressing formed according to the present invention.

FIG. 2 is a bottom view of one alternative aspect of the article, composition, laminate or dressing of FIG. 1.

FIG. 3 is a schematic sectional view of a foam material constructed according to one embodiment of the invention.

FIG. 4 is a schematic sectional view of a foam material constructed according to one alternative embodiment of the invention.

FIG. 5 is a schematic top view of an article, composition, laminate or dressing formed according to a further alternative embodiment of the invention.

FIG. 6 is a schematic sectional view of a foam material having a porosity structured according to one optional embodiment of the present invention.

FIG. 7 is a schematic sectional view of a foam material having a porosity structured according to an alternative embodiment of the present invention

FIG. 8 is a schematic sectional view of an article, composition, laminate or dressing comprising foam according to an additional embodiment of the invention.

### DETAILED DESCRIPTION

Antimicrobial compositions and articles, such as laminates or wound dressings, of the present invention may contain a suitable antimicrobial agent. Any suitable antimicrobial agent or combination of agents can be utilized, such as a polymeric biguanide (e.g., polyhexamethylene biguanide (PHMB) and/or polyethylene hexamethylene biguaide (PEHMB)) and/or ionic metal(s), alone or in combination.

According to further nonlimiting examples, suitable antimicrobial agents include, alone or in combination, certain metals or compounds including such metals, such as silver, gold, copper or zinc may be used as the antimicrobial agent(s). It is additionally contemplated that the antimicrobial treatment could be a combination of a number of agents such as silver, PHMB, CHG, EDTA or other suitable antimicrobials such that a synergistic efficacy is realized.

According to certain embodiments, the antimicrobial agent(s) can comprise a cationic surfactant or a cationic quaternary ammonium compound. Non-limiting examples of such compounds include: benzalkonium chloride; benzethonium chloride; cetrimide; cetylpyridinium chloride; chlorphenoctium amsonate; dequalinium acetate; dequalinimum chloride; domiphen bromide; laurolinium acetate; methylbezethonim chloride; myristyl-gamma-picolinium chloride; ortaphnum chloride; triclobisonum chloride; cetalkonium chloride; dofanium chloride; tetraethylammonum bromide; didecyldimethylammonium chloride; tetraethylammonium bromide; dimethyldiallyl ammonium chloride; p-trialkylamioethyl styrene monomer; and trialkyl(p-vinylbenzyl) ammonium chloride.

According to further embodiments, the antimicrobial agent(s) can comprise a cationic surfactant or a polymeric quaternary ammonium compound. Non-limiting examples of such compounds include: poly(diallyl dimethyl ammonium chloride); poly(3-chloro-2 hydroxypropyl) methacryloxyethyl dimethyl-ammonium chloride; poly(acrylamide-methacryloxyethyl trimethyl-ammonium bromide; poly (butyl acrylate-methacryloxyethyl trimethylammonium bromide; poly(1-methyl-4-vinyl pyridinium bromide); poly(1-methyl-2-vinylpyridinium bromide); and poly(methylacryloxyethyl triethyl ammonium bromide).

According to additional alternative embodiments, the antimicrobial agent(s) can comprise a polyquaternium. Polyquaternium is a neologism used to emphasize the presence of quaternary ammonium centers in the polymer. Polyquaterniums are positively charged, and some have antimicrobial properties. There are currently at least 37 different known polymers under the polyquaternium designation. New polyquanterniums are identified periodically. Different polymers are distinguished by the numerical value that follows the word “polyquaternium.” Thus, the present invention contemplates the possible use of any of the currently known polyquaternium-1 through polyquaternium-37 substances, as well as future polyquanterniums, currently undesignated, falling under the broad definition or categorization noted above.

According to further embodiments, the antimicrobial agent(s) can comprise a cationic antimicrobial peptide, such as e-poly-l-lysine, magainin, cecropins, dermaseptin, pexiganan, iseganan, Oniganan, and defensin.

According to additional alternatives, the antimicrobial agent(s) can comprise amphoteric surfactants, such as include alkyl betaines, dodecyl betaine cocoampho glycinate, and cocamidopropyl betaine.

According to additional alternative embodiments, the antimicrobial agent(s) can comprise bromine based compounds such as poly(4-vinyl-N-alkyl pyridinium bromide); and poly(4-vinyl-N-hexylpyridinium bromide).

An article, composition, laminate or dressing according to the present invention may include one or more of the above-described antimicrobial agent(s) associated with at least one material, such as a foam. Any suitable foam can be utilized. Non-limiting examples include polyurethane foam, or a biomaterial-type foam such as an alginate foam, a hyaluronic acid foam, a bioglass foam, or a collagen foam.

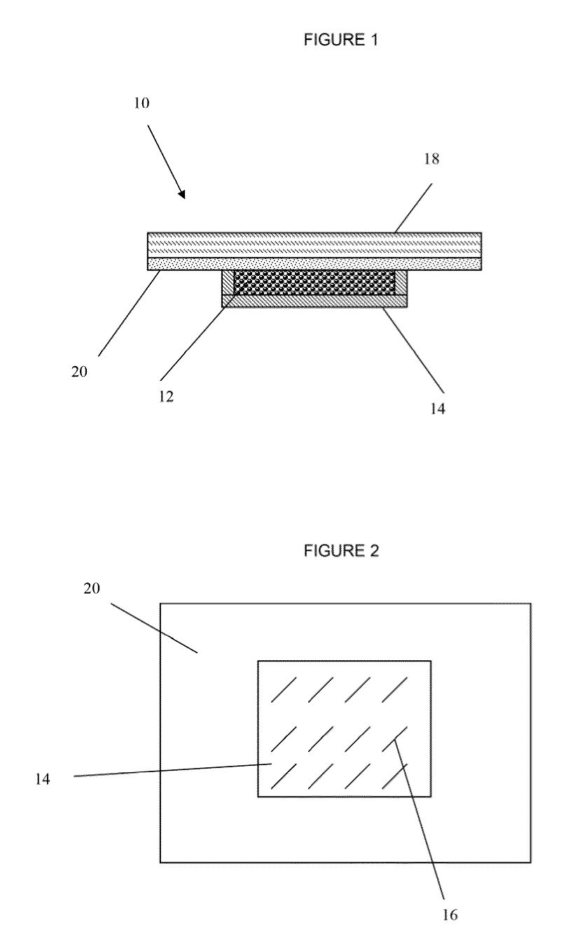

An embodiment of an absorbent article or wound dressing that has antimicrobial properties and keeps moisture away from the patient's skin to prevent maceration, is illustrated in FIGS. 1-2. As shown therein, a laminate or dressing 10 comprises a foam 12 treated with any suitable antimicrobial agent, or combination of agents, such as those materials described above.

The antimicrobial agent may be introduced into the foam 12 in either a liquid form, where it could be part of the aqueous portion of the foam blend, sprayed onto the “green” foam surface(s) prior to oven drying, applied to the dried foam surface(s) by either spray or padding techniques, or in a powder form, where the powder is introduced at the mix-head or sprinkled onto the surface(s) of green foam prior to drying.

According to a further alternative embodiment, foam can be packaged for use soaked in a liquid or a gel containing one or more of antimicrobial agents, optionally with additional other compound(s) of therapeutic value. This liquid or gel would combine with the foam to create an article favorably constructed for wound healing and/or antimicrobial protection.

The agent could also be applied in the form of a film. The dissolution of agent can optionally be controlled by wound fluid amount, pH, ionic strength, organic matter or solubility of excipients or actives in the film. There could be multiple layers of film.

A non-adherent layer 14 made from any suitable material, such as TELFA® or other suitable nonadherent film (e.g., polyolefin, polyester, polyurethane or EVA) is in contact with the wound surface. The non-adherent layer 14 has perforations 16 which allow wound fluid to flow into the foam 12, but separate the moisture from the skin to prevent maceration. The perforations 16 could further be configured such that moisture would be transmitted in only one direction away from the wound. Additionally, the perforations 16 could be adjusted in diameter (from 0.010 to 0.125″) depending on the viscosity of wound fluid being removed. The perforation diameter could be random with a combination to provide variable fluid transmission absorbency performance. The perforation pattern could also be selected with larger perforations in one area of the dressing and smaller in another to control fluid handling performance and minimize adjacent skin maceration. As illustrated in FIG. 1, the non-adherent layer 14 wraps around the edges of the foam 12 to hold the exudate in the chamber formed thereby, isolated from the wound and skin. According to one potential alternative embodiment, an apertured film layer (like those manufactured by Tredegar Corporation, Richmond, Va.) can be used in place of the non-adherent layer 14 to isolate wound fluid from the skin.

The laminate or dressing 10 may further optionally comprise a top film 18 formed from any suitable material, which may be coated with any suitable adhesive 20 to secure the dressing to the skin. The adhesively-backed top film 18 can be breathable to allow moisture to evaporate from the skin, but substantially impermeable to bacteria to prevent contamination. A suitable moisture vapor transmission rate (MVTR) could range from 300-3000 gm/m²/day.

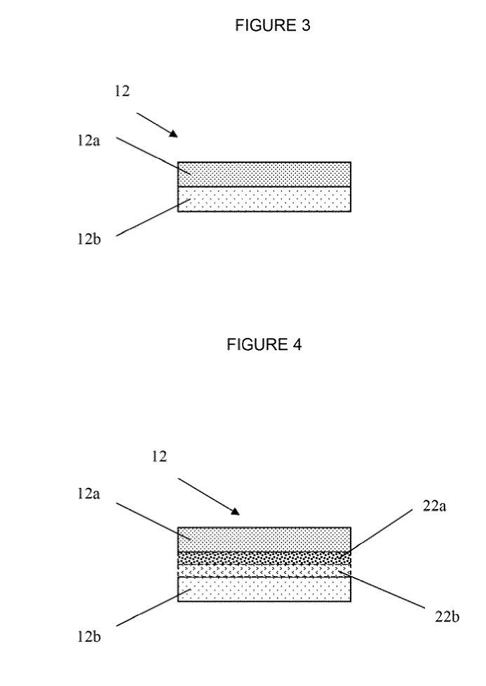

According to a further optional embodiment, the foam 12 can comprise multiple layers of foam of different densities 12a, 12b to direct the fluid absorption in an optimal manner, as shown in FIG. 3. At each interface between foam layers of different densities 12a, 12b, a film 22 (or multiple films 22a, 22b) containing active and/or antimicrobial agent(s) can be inserted. The dissolution behavior of the agent(s) can be controlled via the composition and physical properties of the film(s). One example is the use of transdermal or transmucosal delivery systems, such as dissolvable film technology. Another example is the integration of dissolvable beads into a film form.

Alternatively, the antimicrobial agent(s) can be incorporated into the non-adherent layer 14 layer in addition to, or instead of, the foam 12. According to further alternative embodiments, the non-adherent layer 14 could be coated with a suitable antimicrobial agent or combination of antimicrobial agents, optionally programmed in performance to deliver an effective degree of antimicrobial performance over the expected life of the laminate or dressing 10.

It is additionally contemplated that foam could also be treated with an indicating solution that would remain clear in the presence of the antimicrobial agent, but as the antimicrobial agent is eluted out of the dressing, a change color indicates those areas where the antimicrobial agent(s) is (are) exhausted.

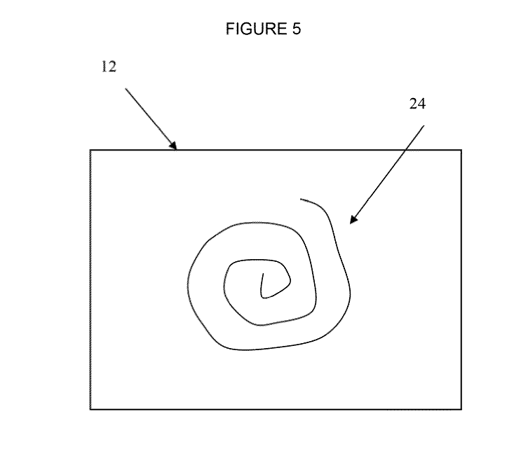

As illustrated in FIG. 5, according to a further embodiment of the present invention, the above-mentioned antimicrobial foam 12 can be provided with centrally located spiral cuts 24. The cuts 24 would be of such a configuration to allow differential swelling of the foam. Such cuts 24 may, for example, allow the wound covering portion of the foam 12 to swell toward the wound instead of causing the foam 2 to buckle and “tent.” Such a feature may therefore provide better fluid management and patient care.

It is also contemplated that the foam structure 12 may contain layers (e.g., 12a, 12b.; FIG. 3) with different swelling properties in terms of speed and swelling ratio. This differential swelling may be designed to cause the foam to buckle and “tent.” Any suitable mechanism can be utilized to promote the desired swelling behavior. One such technique is described in T. Mora et al., “Buckling of Swelling Gels,” Eur. Phys. J. E 20, 119-124, (2006), the entire contents of which is incorporated by reference herein.

Additionally, the absorbent reservoir holding the wound fluid may be periodically pumped out using an externally applied vacuum so that the absorbent reservoir may be reused after it is filled.

According to further optional embodiments, the present invention may comprise a foam composition, a laminate or dressing, wherein the body of the foam presents a gradient of substantially different porosity. The porosity gradient is optionally configured to facilitate controlled release of one or more agents, such as one or more of the above-mentioned suitable antimicrobial agents, contained therein. The agent may be one or more antimicrobial agents, pain management agents, anti-inflammatory agents, debriding agents, wound healing agents, angiogenic factors, scar management agents, or other agents beneficial to wound healing or any combination thereof. For example, a single agent, or combination of different types of the same agent, or combination of two or more different types of agents, may be utilized.

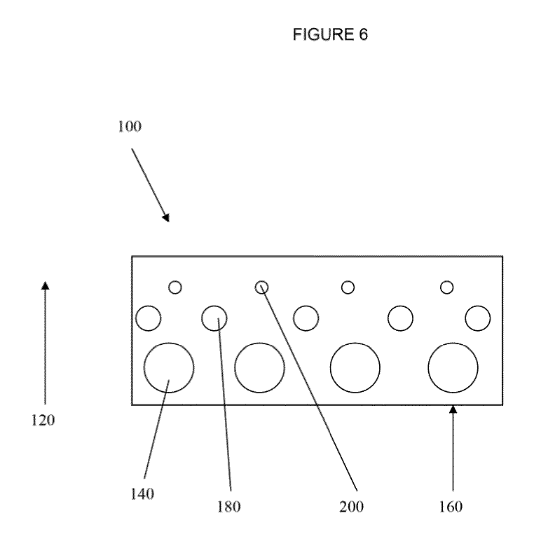

As illustrated in FIG. 6, a foam material 100 having a porosity gradient 120 can be achieved by having larger cells or pores 140 toward the wound-facing side 160 of the material. The foam material 100 can be used, for example, as a wound dressing. The pores may contain one or more antimicrobial agent(s), possibly combined with other therapeutic agents. The pore size decreases (180, 200) further away from the wound-facing side 160 within the foam material 100. The agent(s) may be incorporated into the foam at concentrations of about 0.01%-2% by weight. The larger pores 140 (e.g., about 50-100 μm) at the wound facing side allow a high level of wound fluid into the foam which in turn can causes a correspondingly relatively high level of agent(s) to be released into the wound initially. As more fluid is introduced and travels further into the dressing, the fluid absorption profile is affected by changing cell or pore size 180 (e.g., about 10-50 μm) with a corresponding effect on the release profile of the active agent. Additional changes in pore size 200 (e.g., about 1-10 μm) as fluid travels further up into the dressing, promotes a further change in the fluid absorption and/or active release profiles of the foam 100. Thus, for example, with this configuration a relatively large amount of antimicrobial agent can be released initially with a corresponding relative large absorption of wound exudate, with relatively smaller amounts of agent(s) released subsequently, and the correspondingly relatively smaller absorption of exudate.

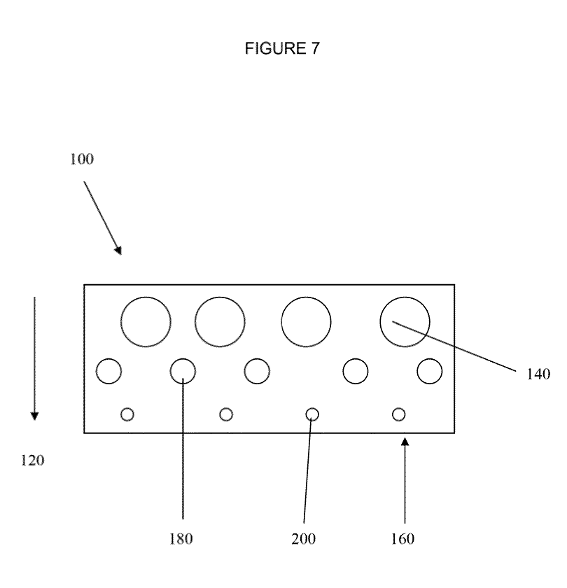

In another aspect of the invention, the absorption and agent release profiles can be reversed relative to the embodiment illustrated in FIG. 6. Thus, in the embodiment illustrated in FIG. 7, the gradient 120 is such that smaller cells or pores 200 are located toward the wound contacting side 160 of the foam material 100, while the pores get larger (180, 140) further away from the wound-facing side 160 within the foam material 100. The smaller pores 200 (e.g., about 1-10 μm) at the wound facing side 160 allow a relatively low amount of wound fluid into the foam initially which in turn can causes a corresponding relatively low level of agent(s) contained within the pores to be released into the wound initially. As more fluid is introduced and travels further into the dressing, the fluid absorption profile is affected by changing cell or pore size 180 (e.g., about 10-50 μm) with a corresponding effect on the release profile of the active agent. Additional changes in pore size 140 (e.g., about 50-100 μm) as fluid travels further up into the dressing, promotes a further change in the fluid absorption and/or active release profiles of the foam 100. Thus, for example, with this configuration a relatively small amount of antimicrobial agent(s) can be released initially along with a correspondingly relative small absorption of wound exudate, with relatively larger amounts of agent(s) released subsequently, and the correspondingly relatively larger absorption of exudate.

While the above-described porosity gradients have been described as being provided within a single foam layer, it is also possible to achieve a similar configuration by attaching two or more different cured foams of different porosities and/or thickness together via casting, extrusion or appropriate adhesive systems

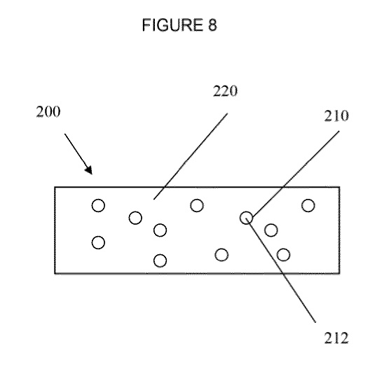

As illustrated in FIG. 8, according to further aspects of the present invention, a material 200 is provided having dissolvable beads 210 (e.g., phosphate glass, starch particles or others) are incorporated into a foam matrix 220. Material 200 is suitable for use as a wound dressing. Beads 210 may encapsulate any suitable antimicrobial agent(s), such as those mentioned above, and may also contain one or more of the other therapeutic agents disclosed herein, collectively identified as element 212 in FIG. 8. The beads 210 may be provided in a “programmable” sequence. For example, more fluid means more beads dissolve and create more open spaces to hold more fluid, conversely less fluid causes less beads to dissolve and thus create fewer open spaces, thereby helping to maintain ideal fluid equilibrium at the wound site for optimum moist wound healing environment. Examples of suitable bead sequences or distributions can include density gradients of similar sized beads, or used of beads of differing sizes, as illustrated in FIG. 6-7. For example, as the beads 210 dissolve, voids are left behind. Such voids can increase absorbency over time. Thus, the size of dissolving beads 210, and their distribution pattern within the foam matrix can be selected so as to increase or decrease absorbency over time by a desired degree, as was explained above. The beads 210 may optionally be incorporated into foam in a pattern to control direction and nature of swelling of a laminate or dressing as it absorbs wound exudate.

The above-mentioned antimicrobial contained beads can be formed by any suitable technique. For example, the antimicrobial agent(s) can be mixed with in supercritical carbon dioxide, and then this mixture is placed under pressure, during polymerization or polymer formation. Controlled dosing of carbon dioxide will control polymer viscosity. By suddenly reducing pressure carbon dioxide expands to form nanobubbles of antimicorbial agent(s) in the polymer matrix.

In yet another embodiment of the present invention, in a controlled release foam material described herein, salts such as sodium chloride, and/or potassium chloride and/or EDTA are used to achieve a porosity gradient within the foam matrix similar to that shown in FIG. 6 or 7, or described above. The gradient is achieved by controlling the size of the salt crystals within the foam matrix. Suitable sizes may include the pore size ranges described above. The active agent may be incorporated as salt only, into the foam only, or both in the foam and the salt. As fluid is absorbed into the foam matrix, the salt is dissolved and an open void is left behind. The amount of active agent in the salt, the size of the salt crystal, and the size of the void left by the dissolved salt crystal control and define the release profile of the active agent, as well as the absorption profile by the void or pore left behind. Differences in solubilities of salts can be used to achieve varied porosity or a channels of pores. For example, use of a combination of salt with low and high solubility in wound fluid. Highly soluble salt would dissolve creating a network of pores first while low solubility salt area would remain as is or slowly create a network of pores.

In yet another embodiment, sodium/calcium alginate particles are treated with antimicrobial and/or therapeutic agent(s) and are strategically distributed within the foam structure. The density may vary within the foam matrix, and/or the size of the particles may vary within the material, in any manner such as those illustrated and described herein. Pores or voids are created in the foam as these particles are jelled and liquefied, such as upon contact with wound exudate. Thus, according to one optional use of the material, mechanisms of the type described herein for programmed release of antimicrobial and/or therapeutic agent(s), as well as for programmed moisture transfer away from the wound.

According to further optional embodiments, PHMB encapsulated nanofibers or spheres may be incorporated into a foam matrix. According to further alternative embodiments, non-dissolvable nanospheres, nanoparticles, or beads could be placed in the foam matrix, along with dissolvable particles that could create porosity upon dissolution by wound fluid absorbed. These non-dissolvable nanospheres, nanoparticles, or beads could function as to remove or inhibit function of undesirable elements from wound fluid by any suitable mechanism. Suitable mechanisms include selective binding techniques, either directly or through an intermediary substance already attached to the nanospheres, nanoparticles, or beads.

Accordingly to further embodiments, foam layers of different porosities and/or properties are layered in “green” uncured stage of foam formation. Alternatively, it is also possible to attach two different cured foams of different porosities and/or thickness together via appropriate adhesive systems.

According to a further embodiment, dissolvable alginate or carboxymethylcellulose fibers are included in the foam, laminate or dressing to control swelling. The fibers may optionally be directionally oriented so as to control swelling in a desired direction. For example, the imbedded fibers become securely attached and part of the foam after the curing and drying process. Due to this attachment the fibers provide a resistance to movement of the foam along the fiber axis during foam hydration. A bi-axial or multi-axial orientation of these fibers would provide a planar resistance to the foam swelling, thereby encouraging swell perpendicular to the fiber plane. The fibers may take a variety of forms such as, but not limited to, a woven or non-woven structure or a plurality of individual, continuous fibers.

According to a further alternative embodiment, nontoxic chemical particles which would control pH of wound for optimum healing such as sodium bicarbonate, citric acid salts, etc. are included in the foam, laminate or dressing.

Wound dressings can, of course, include additional active ingredients or agents such as, for example, a therapeutic agent, an organoleptic agent, a growth factor, an analgesic, a tissue scaffolding agent, a haemostatic agent, a protein inhibitor, collagen, enzymes, an anti-thrombogenic agent, an anesthetic, an anti-inflammatory agent, an anticancer agent, a vasodilation substance, a wound healing agent, an angiogenic agent, an angiostatic agent, an immune boosting agent, a skin sealing agent, an agent to induce directional bacterial growth, an agent to impart bactericidal or bacteriostatic activity, an electron transfer agent to destabilize or destroy the metabolic action of microbes and/or biofilm formation, combinations thereof and the like. Release of active agents may be triggered by a variety of means, such as, for example, an electric field or signal, temperature, time, pressure, moisture, light (e.g., ultra-violet light), ultrasound energy, sonication, combinations thereof and the like.

Any numbers expressing quantities of ingredients, constituents, reaction conditions, and so forth used in the specification are to be understood as being modified in all instances by the term “about”. Notwithstanding that the numerical ranges and parameters setting forth, the broad scope of the subject matter presented herein are approximations, the numerical values set forth are indicated as precisely as possible. Any numerical value, however, may inherently contain certain errors or inaccuracies as evident from the standard deviation found in their respective measurement techniques. None of the features recited herein should be interpreted as invoking 35 U.S.C.§112, ¶6, unless the term “means” is explicitly used.

Although the present invention has been described in connection with preferred embodiments thereof, it will be appreciated by those skilled in the art that additions, deletions, modifications, and substitutions not specifically described may be made without departing from the spirit and scope of the invention.

We claim:

1. An article comprising:
at least one layer of foam, the foam layer and at least one antimicrobial agent associated with foam layer, the antimicrobial agent comprising PHMB, PEHMB, or derivatives thereof;
at least one non-adherent layer disposed on at least a portion of the foam layer, the non-adherent layer being permeable to moisture; and
a film disposed on at least another portion of the foam layer, the film being breathable to allow escape of moisture, but substantially impermeable to bacteria.

2. The article of claim 1, wherein the article comprises a wound dressing, the wound dressing having a wound facing side formed at least in part by the non-adherent film.

3. The article of claim 1, further comprising multiple layers of foam of different densities.

4. The article of claim 3, wherein at least one interface is formed between the multiple layers of foam, and further comprising at least one layer containing at least one antimicrobial agent disposed at the interface.

5. The article of claim 1, further comprising at least one antimicrobial agent associated with the non-adherent layer.

6. The article of claim 1, wherein the at least one foam layer further comprises an indicating agent capable of visually indicating the absence of antimicrobial agent in the foam layer.

7. The article of claim 1, wherein the foam comprises a polyurethane foam, an alginate foam, a hyaluronic acid foam, a bioglass foam, or a collagen foam.

8. The article of claim 1, wherein the foam where is surrounded on at least three sides by the non-adherent layer.

9. The article of claim 1, wherein the one non-adherent layer comprises a polyolefin, a polyester, a polyurethane, or EVA.

10. The article of claim 1, wherein the non-adherent layer is structured so as to permit the transmission of moisture in one direction only, the one direction being in the direction of the foam layer.

11. The article of claim 1, wherein and not adherent layer is perforated.

12. The article of claim 1, further comprising an adhesive disposed on at least a portion of the film.

13. The article of claim 1, wherein the film comprises a moisture vapor transmission rate of about 300-3000 gm/m²/day.

14. The article of claim 1, wherein the foam where comprises a spiral cut to promote differential swelling of the foam upon absorption of moisture therein.

15. The article of claim 1, wherein the at least one foam layer comprises a first foam layer in the second foam layer, the first foam layer having swelling properties upon absorption of moisture therein that is different than the swelling properties of the second foam layer, such that upon absorption of moisture therein the foam buckles or tents.

16. An article comprising at least one layer of foam, the foam comprising pores of different sizes, at least some of the pores at least partially filled with at least one elutable antimicrobial agent, the pores of different sizes forming a gradient with the foam layer.

17. The article of claim 16, comprising about 0.01%-2% by weight antimicrobial agent.

18. The article of claim 16, wherein the foam comprises a polyurethane foam, an alginate foam, a hyaluronic acid foam, a bioglass foam, or a collagen foam.

19. The article of claim 16, wherein the antimicrobial agent comprises: a polymeric biguanide; a cationic quaternary ammonium compound; a polymeric quaternary ammonium compound; a polyquaternium; a cationic antimicrobial peptide; or combinations thereof.

20. The article of claim 16, wherein the article comprises a wound dressing, the foam having a wound facing side, wherein the gradient comprises relatively larger pores disposed proximate to the wound-facing size, and relatively smaller pores within the foam further away from the wound-facing side.

21. The article of claim 16, wherein the pores define an essentially trimodal pore size distribution of pores having average pores size ranges of about 50-100 μm, about 10-50 μm, and about 1-10 μm.

22. The article of claim 16, wherein the article comprises a wound dressing, the foam having a wound facing side, wherein the gradient comprises relatively smaller pores disposed proximate to the wound-facing size, and relatively larger pores within the foam further away from the wound-facing side.

23. The article of claim 20, wherein the pores define an essentially trimodal pore size distribution of pores having average pores size ranges of about 50-100 μm, about 10-50 μm, and about 1-10 μm.

24. An article comprising: a foam matrix and a plurality of dissolvable members disposed with the foam matrix, at least one antimicrobial agents associated with the dissolvable members such that upon dissolution thereof the antimicrobial agent is eluted and pores or voids are created in the foam matrix.

25. The article of claim 24, wherein the foam comprises a polyurethane foam, an alginate foam, a hyaluronic acid foam, a bioglass foam, or a collagen foam.

26. The article of claim 24, wherein the antimicrobial agent comprises: a polymeric biguanide; a cationic quaternary ammonium compound; a polymeric quaternary ammonium compound; a polyquaternium; a cationic antimicrobial peptide; or combinations thereof.

27. The article of claim 24, wherein the dissolvable members comprise: glass beads, starch particles, salt crystals, or alginate particles.

28. The article of claim 24 further comprising non-dissolvable members disposed in the foam matrix.
